(19) United States
(12) Patent Application Publication
(10) Pub. No.: US 2010/0239625 A1
(43) Pub. Date: Sep. 23, 2010

(54) TRANSPARENT ANTIMICROBIAL FACE MASK

(76) Inventors: Anne McIntosh Puckett, Mooresville, NC (US); Melvin Glenn Mitchell, Penrose, NC (US)

Correspondence Address: ADAMS INTELLECTUAL PROPERTY LAW, Suite 2350 Charlotte Plaza, 201 South College Street, CHARLOTTE, NC, 28244, US

(21) Appl. No.: 12/303,775

(22) PCT Filed: Feb. 29, 2008
(86) PCT No.: PCT/US08/55497

§ 371 Date: Dec. 8, 2008

Related U.S. Application Data
(60) Provisional application No. 60/892,700, filed on Mar. 2, 2007.

(51) Int. Cl. A61F 13/12

(52) U.S. Cl. 424/402

(57) ABSTRACT

A mask for being worn over the nose and mouth of a wearer, and a panel having sufficient transparency to permit visual observation of the mouth of the wearer. The panel is formed from first and second overlying transparent, pleated films perforated with elongate slits sized to permit two-way transit of gases, but not liquids, under atmospheric pressure. The first and second films each include a multitude of projections for maintaining between the first and second films a multitude of tortuous flow paths for permitting respiratory gas flow through the panel.

### TECHNICAL FIELD AND BACKGROUND OF THE INVENTION

Face masks are universally used in medical, industrial, home, consumer and other applications to reduce the risk of transferring infectious bacteria, viruses and the like between the wearer and other individuals or with the environment. Historically, such face masks extend from the bridge of the nose to the chin, and include a plurality of layers of opaque woven or nonwoven filter media that totally block the ability to see the mouth and nose of the wearer. The insulating effects of the mask and the inability to see the mouth of the wearer hinder direct communication and the speech quality of the wearer. Still another disadvantage of the opaque mask is that it hides the reassuring mouth and facial movements of the wearer, its use is socially uncomfortable and subject to misinterpretation and/or personal separation in many situations. In addition, these face masks only provide one-way protection and, even then, relatively low protection. These limitations dramatically restrict the widespread use of face masks in medical and non-medical settings, e.g.; law enforcement, public transportation, military, and the like.

Research in the field of speech communication has long revealed that 65 percent of personal communication messages comes from non-verbal cues (which includes facial expressions) while only 35 percent of the message stems from the actual verbal content of the speech. There is also general consensus that when interacting with small children who may not understand all that is being said, the ability to see a person's face clearly is more reassuring and aids in comprehension and imitative learning. Lastly, research from the speech-language pathology field shows that all people (normal-hearing as well as hearing-impaired) do a certain amount of speech-reading (e.g.: lip reading).

Face masks that use nonwoven filter media are, in most cases either permeable to liquids or, at best, resistant to fluids that come in contact with either side of the permeable portion of the mask. Finally, face masks that use a plurality of layers of opaque nonwoven filter media typically provide one-way protection from the healthy person, e.g., health care provider, not the sick person. Thus, a transparent face mask that provides two-way microbial protection, repels fluids and may optionally be treated with one or more antimicrobial agents to enhance its ability to reduce the transmission of pathogens would be highly desirable in numerous contexts (e.g.: preschool settings, schools, hospitals, offices with lots of cubicles, airline travel, subway, or other enclosed spaces with large numbers of people). Such a mask could reduce the likelihood of illness and disease since it would be intended to kill most pathogens on contact.

The development of a suitable transparent mask would also improve communication and understanding between the wearer and non-wearer alike. The non-porous border or framing fabric can also be used as a carrier for graphic art that would relax the non wearer, e.g.; colors, designs, cartoons, animals, flowers, and the like for certain segments of the population to create a less “medical” appearance in the mask and make widespread use more socially comforting, thereby reducing transmission and contamination with airborne pathogens. Masks in use in the Far East take advantage of this feature to make wearing a mast more socially acceptable.

### SUMMARY OF THE INVENTION

One aspect of the invention relates to a medical face mask which is fabricated of a highly porous transparent film framed by a nonporous edge onto which is attached a nose clip and head straps. The highly porous transparent film may optionally be treated with one or more antimicrobial, anti-reflective, and/or anti-fog coatings. The porosity of the mask is created by engineered openings having particular shapes that permit the passage of gases and prevent the passage of microorganisms. In addition, materials may be used on the mask that are intended to kill micro-organisms on contact.

It is a more general object to describe both the process and product which provide singular and plural micro-vented laminae that will permit controlled passage of a gas without passage of a liquid, controlled passage of gas and liquid from one side to another of the laminae, but in the reverse direction, permit controlled passage of a gas without passage of a liquid (one way valve), and controlled passage of gas and liquid from one side to another of the laminae.

Fabrication of the singular and plural micro-vented laminae includes the steps of forming a nonporous web substrate, micro-venting the nonporous web substrate with vents in which the total open orifice area comprises about 0.1% to 95.0% of the total surface area of the layer.

More generally, the films, foils, webs, nonwovens, laminates and sheets are adaptable for many uses, including absorbent back sheets, adult incontinent garments, baby diapers, and feminine hygiene products; wound dressings, breathable water proof bandages, and artificial skin; agricultural/horticultural covers, row crop covers, tarpaulins, greenhouse covers; construction wraps, house wrap, replacement for extensible kraft on fiber glass insulation, roofing barriers; breathable storage cases, gun cases, duffle bags, back packs, tent or portable shelter storage bags; recreational or military protective coverings, tents, tarpaulins, shelters; agricultural/horticultural packaging such as produce packaging, live plants, live animals such as chickens, reptiles, amphibians and the like; breathable storage and transport cases for aquatic life, including fish, plants, lobsters, invertebrates where oxygen can migrate into the water for aeration; aqualungs, helmets or containers with filtration systems for underwater containment of living organisms which require oxygen and CO₂ diffusion across the membrane; attached or floatable breathing filtration system for snorkeling; filtration systems for submarines; medical/biological membranes for nourishment or protection; packaging for medical devices; chemical reaction membranes for gas diffusion into liquids; liquid waste treatment aeration system for introduction of air for reduction of BOD; processing aid for lamination of two or more previously nonporous webs with water or solvent based adhesives; liners, clothing liners, footwear liners, casket liners, water proof gloves, waterproof socks, hats, hoods; surgical garments and barriers where breathing is an advantage for comfort yet sterile protection from liquids; drapes, surgical gowns, surgical caps, plastic medical barriers; geo-textile barriers; and landfill barriers.

The films, foils, webs, nonwovens, laminates and sheets are also applicable to a wide range of clothing and apparel items, including rain, sports and athletic clothing, such as coats, pants, hats and undergarments, and industrial garments where breathing is an advantage for comfort, yet protection from hazardous liquids is necessary or desirable.

The films, foils, webs, nonwovens, laminates and sheets also have application in desiccant packaging; food packaging evaporation/concentration containers, packages or pipes to allow liquid vapor removal, therefore increasing concentration of other contents liquid or solid; porous packaging which will allow filling with ingredients while allowing air to be displaced through membranes; overcover protective membranes for air release as wall coverings, equipment coverings, adhesive labels and the like are being covered.

The films, foils, webs, nonwovens, laminates and sheets are also suitable as waterproof and water resistant barriers with reduced wind drag; signage, banners, walls; humidification containers, packaging or piping; slow release watering devices for plants; cook-in bags for off-gassing, browning and moisture removal without pressurization; automotive coverings: convertible tops and car covers. Additional medical applications include radially perforated contact lenses for improved breathability and blood oxygenation systems.

### BRIEF DESCRIPTION OF THE DRAWINGS

Some of the objects of the invention have been set forth above. Other objects and advantages of the invention will appear as the description of the invention proceeds when taken in conjunction with the following drawings, in which:

FIG. 1 is a elevational view of a single layer mask;

FIG. 2 is an elevational view of a double layer mask; and

FIG. 3 is a cross section of a double layer mask showing contact between the two film layers.

### DESCRIPTION OF THE PREFERRED EMBODIMENTS AND BEST MODE

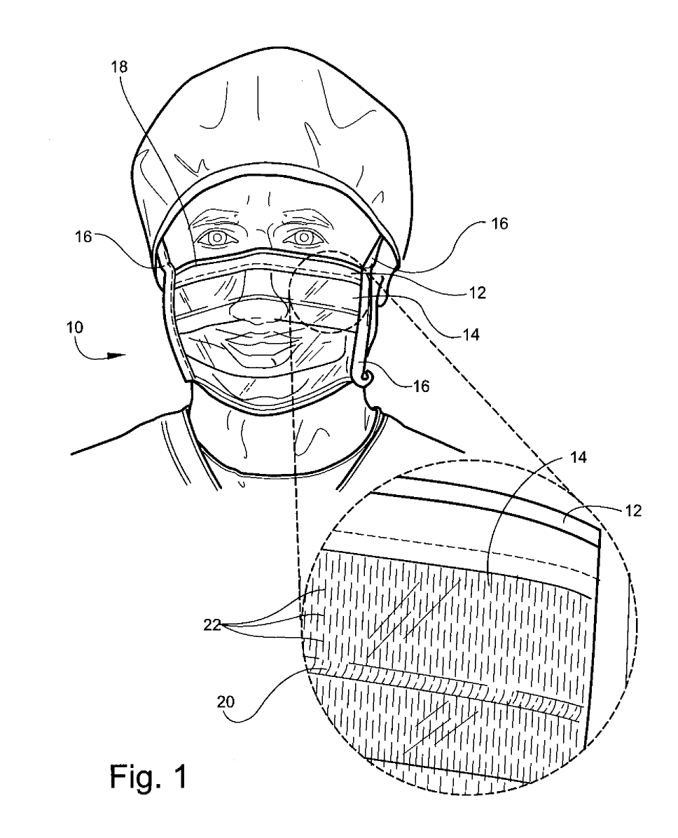

Referring now specifically to the drawings, a transparent, antimicrobial face mask according to the present invention is shown generally in FIG. 1 at reference numeral 10. The mask 10 includes a frame 12 into which is attached by any suitable means to a transparent panel 14. The mask 10 includes suitable straps 16 that extend behind the head of the wearer and retain the mask 10 in place over the nose and mouth of the wearer. The mask may also include a nose clip 18 that is sufficiently deformable to permit the top of the mask 10 to be closely conformed to the nose and adjacent facial structures.

In a typical flat face mask design pleats 20 are included in the transparent filter media to provide facial flexibility for improved fit and greater surface area for lower pressure drop per breath. The pleats 20 may be vertically or horizontally oriented. In addition, different size masks (S, M, L, XL) can be offered for improved fit, comfort and feel.

There are several specific technologies and steps associated with the process of forming a highly porous transparent film with simultaneous liquid retention and gas/vapor venting characteristics for use in a transparent face mask.

The transparent panel 14 is formed from a thermoplastic film selected from the group of thermoplastic films including polypropylene (PP), polyethylene (PE), polyethylene terephthalate (PET), nylon 6 (N6), nylon 66 (N6,6), polytetrafluoroethylene (PTFE), polyvinyifluoride (PVF), polyvinylchloride (PVC), polyvinylidenefluoride (PVDF), and polyvinylidenechloride (PVDC).

The panel 14 is perforated with slits 22 preferably having a length from 1 to 5 mm, and holes having a diameter of no more than about 0.127 mm, or 5 mils.

The slits 22 are spaced from each other so that the perforations define a total open orifice area of approximately 30% to 95.0% of the total surface area of the layer. The film may optionally be treated with one or more antimicrobial, anti-reflective, and anti-fog coatings. The mask 10 is intended to be used only once and then disposed of in a suitable manner.

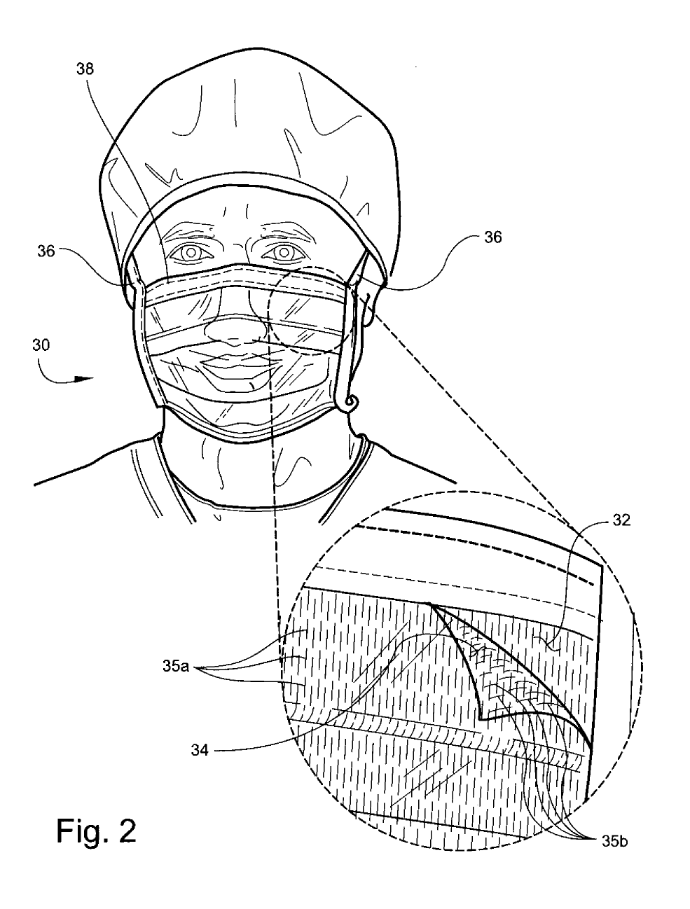

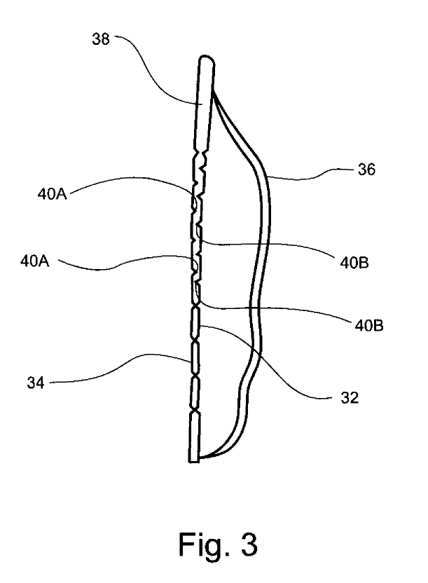

Referring now to FIGS. 2 and 3, a double layer mask 30 is shown and includes a panel formed of a pair of overlaid filtration films 32, 34, and suitable straps 36 that extend behind the head of the wearer and retain the mask 30 in place over the nose and mouth of the wearer, as described above with reference to FIG. 1. The mask 30 may also include a nose clip 38 that is sufficiently deformable to permit the top of the mask 30 to be closely conformed to the nose and adjacent facial structures.

In the preferred embodiment disclosed, at least one of the films 32, 34 is provided with slit or holes 35A, 35B, and further, is embossed to provide a multitude of separated projections 40A, 40B that face inwardly. The projections 40 stand proud of the major surface of at least one of the films 32, 34 and allow the films 32, 34 to contact each other in a multitude of contact locations at the projections 40A, 40B for maintaining between the first and second films 32, 34 and the contact locations a large number of irregularly-shaped flow paths that permit respiratory gas flow in both directions through the panel. It is not necessary to align the slits or holes. Spacing between the films 32, 34 between the projections 40A, 40B allow gases to flow into the slits or holes in one of the films 32 or 34, travel laterally in the voids between the contacting projections 40A, 40B. The preferred manner of forming the projections 40A, 40B is by embossing, but punching holes to leave hanging chads is also suitable, as discussed further herein.

Most conventional antimicrobials that are legal for use in this environment, including quats, bleach, peroxides, phenols, triclyosan, formaldehydes, silver ions and synthetic toxic formulations, and the like, work on the basis of diffusion of the biocide away from the treated surface. This promotes microbial adaptation, loss of effectiveness, leaching, and diffusion.

Only one known biocide offers a purely mechanical contact kill mechanism: organosilane molecules. This antimicrobial product offers a preferred embodiment for treatment of the highly porous film. It is available as a stable aqueous solution that can produce a durable microbiostatic coating on a broad range of textiles and films.

A conventional quaternary ammonium salt (organo) is chemically spliced to a silane molecule, resulting in a highly active molecule (3-trimethoxysilylpropyldimethyloctadecyl ammonium chloride) that has both tenacious bonding capabilities as well as excellent antimicrobial properties. Once applied to a target surface for use in construction of a transparent face mask, it initially bonds to the surface on all available receptor sites (principally H+).

Afterward, stable bonds between remaining OH— sites on the molecule and the positive charge on the nitrogen atoms (N+) form, resulting in the creation of a large co-polymer involving the target and the organosilane. Since there is no unfixed organosilane once the water evaporates, there is no dislodgeable residue and no odor, leaching, off-gassing, migration or diffusion of the molecule.

The molecular structure of the organosilane compound is set out below:

### Fabrication and Wear Prototype Examples

### Powerful Combination (SiQac)

### Organosilane Quaternary Amino

### Molecular Structure:

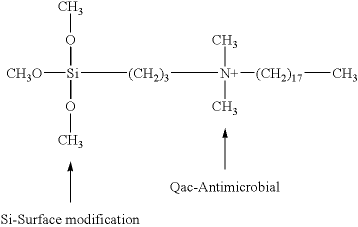

### Example 1

A transparent face mask was fabricated based on the general physical structure of a 3M Nexcare Earloop Mask. A 3M Nexcare Earloop Mask measures roughly 3¼ inches×6¾ inches as removed from the box. The filter area comprises three nonwoven polyolefin layers. There are two poorly formed point-bonded spunbonded outer layers weighing 22 gsm (0.67 oz/yd²) each: one blue (to outside) and one white (to inside) and a meltbrown interior layer weighing 13 gsm (0.40 oz/yd²). The filtration area before pleat expansion measures 2¼ inches×5½ inches. With the pleats expanded (stretched out), the filtration area measures 5½ inches×5½ inches. The indentations average ˜⅝ inch in each direction per pleat. The pleats provide two attributes: Facial flexibility for improved fit and greater surface area for lower pressure drop per breath. The total surface area available for filtration is 30¼ in² per mask.

A typical human nostril can be assumed to be roughly 9 mm to 13 mm in diameter. Typical total nostril orifice area for two paired nostrils, not counting irregularities, would be roughly 170 mm² (˜0.25 in²). A typical human mouth open and breathing in a non-athletically exerted fashion can be assumed to be roughly 38 mm wide by 13 mm high. Typical open mouth orifice size when breathing, not counting irregularities, would be roughly 250 mm² or ˜0.4 in². A typical volume of a single breath under normal non-exertion conditions fills a sphere at atmospheric pressure that is roughly 5 cm in diameter. Volume of a Sphere, V=4/3*pi*r³; therefore a typical breath has a volume of approximately 65 ccm. A typical breath while talking requires almost double the volume of breath needed for breathing in order to allow verbalization, or roughly 120-130 cm³.

A sheet of 48 gauge nylon film was cut 7 inch×7 inch. The film was pleated similar to the polyolefin spunbond/meltblown/spunbond layers described above with pleats held in place with transparent tape. Next, the material of the filtration area was removed from a 3M Nexcare Earloop Mask. Hot melt was then placed around the remaining circumference of the outside of the mask which surrounds the removed rectangle that measures 2¼ inches×5½ inches with the outside remaining structure being ˜½ inches to ⅝ inches wide. The 7 inch×7 inch pleated nylon film is then pressed firmly into the hot melt, the pleats are filled with hot melt on the ends, the constructed mask is inspected for complete seal (air tightness) and resealed as necessary.

Before donning of the micro-vented mask, an unaltered 3M Nexcare Earloop Mask was worn for a time to obtain a feedback for wear and feel.

The 5½ inches×5½ inches nylon film filtration area installed onto the 3M Nexcare Earloop Mask perimeter is then micro-vented using a 2 mm length slit pattern. Five densities of these 2 mm length slit patterns are prepared: 30 vents/in²; 50 vents/in²; 70 vents/in²; 90 vents/in² and 160 vents/in².

General math surrounding mask vent specifics follow:

2 mm length slit pattern=Circumference, C=2*pi*r

Radius of slit once open, r=0.637 mm, r=C/pi*r

Orifice area of slit once open, A=1.274 mm, A=pi*r²

645 mm²=1 in²

3M Nexcare Earloop Mask Surface Area=30.25 in²

The transparent face mask thus prepared was then worn for at least ˜15 minutes to obtain feedback for wear and feel. A table comparing fit and feel data to orifice density/open orifice area follows:

TABLE I
Transparent Face Mask Wear & Feel Data
(2 mm Length Slit Pattern Micro-Vent)

| | Total | Percent | Total | |
|---|---|---|---|---|
| | Open | Open | Open | |
| Density | Orifice | Area | Orifice | |
| (vents/inch2) | (mm²) | (%) | (inch²) | Wear/Feel Comments |
| 0 | 0 | 0 | 0 | Suffocation |
| 30 | 38 | 5.9 | 1.8 | Pulls in & out with |
| | | | | breath/nose breathing |
| | | | | comfortable/mouth |
| | | | | breathing marginally |
| | | | | comfortable |
| 50 | 64 | 9.9 | 3.0 | No in & out with breath/ |
| | | | | nose & mouth |
| | | | | breathing comfortable |
| | | | | except w/talking |
| 70 | 89 | 13.8 | 4.2 | Nose & Mouth breathing |
| | | | | comfortable while |
| | | | | talking |
| 90 | 115 | 17.8 | 5.4 | Nose & Mouth breathing |
| | | | | comfortable except |
| | | | | during physical exertion |
| 160 | 204 | 31.6 | 9.6 | Nose & Mouth breathing |
| | | | | comfortable during |
| | | | | physical exertion |

The 5½ inches×5½ inches nylon film filtration area installed onto the 3M Nexcare Earloop Mask perimeter was then micro-vented using a 2.6 mm length slit pattern. One density of this 2.6 mm length slit pattern was prepared: 160 vents/in². General math surrounding mask vent specifics follow:

2.6 mm length slit pattern=Circumference, C=2*pi*r

Radius of slit once open, r=0.828 mm, r=C/pi*r

Orifice area of slit once open, A=2.153 mm, A=pi*r²

645 mm²=1 in²

3M Nexcare Earloop Mask Surface Area=30.25 in²

The transparent face mask thus prepared is then worn for at least ˜15 minutes to obtain feedback for wear and feel. A table comparing fit and feel data to orifice density/open orifice area follows:

TABLE II
Transparent Face Mask Wear & Feel Data
(2.6 mm Length Slit Pattern Micro-Vent)

| | Total | Percent | Total | |
|---|---|---|---|---|
| | Open | Open | Open | |
| Density | Orifice | Area | Orifice | Wear/Feel |
| (vents/inch2) | (mm²) | (%) | (inch²) | Comments |
| 160 | 344 | 53.4 | 16.2 | Nose & Mouth |
| | | | | breathing |
| | | | | comfortable during |
| | | | | physical exertion |
| | | | | Vents are more |
| | | | | visible on mask |

A transparent face mask prototype has been developed around the general design of a 3M Nexcare Earloop Mask with the opaque nonwoven filtration media replaced by a single micro-vented 48 gauge nylon film. Although a 48 gauge or 0.00048 inch thickness nylon film was used in the current examples, the described technology's functionality applies equally to most film thicknesses and compositions. The following broad conclusions are gleaned from the subject work to date:

1) Talking requires air exchange almost equal to general exercise when wearing face mask. Filtration open orifice area must be significantly larger 5× to 10× than both nose and mouth orifice to achieve comfort with single layer high porous film.

2) Transparent face masks using a single nylon film and 2 mm length slit pattern micro-vent must have open orifice size area greater than 30% for comfort.

3) The larger the open orifice area the more robust the conditions under which the transparent mask may be worn.

The preferred embodiment for single layer high porous film design for construction of a transparent face mask is to use as high a vent count as possible, e.g.; 160 vents/in².

Visibility of the mouth can be enhanced by placing pleats both above and below the lip with unpleated film directly in front of the mouth. Several product designs that were attempted produced less desirable results: the impact of a larger slit (2.6 mm length slit pattern micro-vent) provides much improved wear/feel comfort, but the longer vents tend to become more visible to the eye, negatively impacting transparency. The impact of a extremely light weight 8.5 g/mm² transparent nylon flatbond spunbond Cerex Type 23 used as a diffusion/comfort/bacteria spacer layer between mouth and micro-vent film was evaluated. Use of this substrate between film and mouth appears to improve wear/feel comfort. Unfortunately, if a pleat were to become positioned over the mouth, the mask transparency is greatly reduced and becomes opaque, resulting in the inability to see the mouth of the wearer. In all the examples used, microvents are the preferred embodiment as the design lets air but not liquid pass. A highly porous film using very large holes that if used in multiple layers as the mask is constructed can block direct air flow yet be breathable and clear. For example, a sheet of nylon perforated with a standard 6 mm diameter hole punch leaves dimpled chads which, when used in conjunction with multiple sheets, will contact the sheets at spaced distances from each other and thereby space the sheets to provide a tortuous path for the air to pass. The holes between the two sheets are preferably positioned such that they do not align. Any highly porous film that is safe for human use which, when used either singularly or in multiple layers to construct the mask that the design is suitable. Multiple layers that are not as transparent as a single layer, are still suitable. Two layers are preferred, but more layers can be used depending on overall transparency.

In all of the examples, “microvents” are the preferred embodiment as the design lets air but not liquid pass.

For the PE with 7 (3 mm) vents/inch per & 16 lines/inch or 112 vents/inch² or 50% open area, the embossing spacing is on 2 mm centers in MD & CD. For larger vents, the spacing can be greater or at least equal to the length of the vent. Nominal outwardly projection of the embossed areas is 0.25 to 1.00 mm.

The preferred embodiment contains 2-4 layers, with the most preferred embodiment containing 2 layers. More broadly, masks according to the invention may contain from 1-10 layers, though the more layers used, the poorer the visibility through the mask.

### Laboratory Analysis Data:

Bacterial Filtration Efficiency BFE is a measure of the material's ability to prevent the passage of bacteria. Experimental testing was done by preparing a Staphylococcus aureus solution and introducing the solution to the experimental materials. The BFE represents the efficiency of the experimental materials from penetrating the mask surface. A higher BFE represents a more efficient and desirable mask. In experiment testing, it was determined that a 1 mil PE Single Film had a BFE rating of approximately 18%. A 1 mil PE film combined with a ½ mil PE Film (Double Film) had a BFE rating of approximately 28%.

Using a Markson Science—“% Transmission Equipment” set using a 660 Nanometer light source, the following data was obtained for light % Transmission through various potential designs of film layers useful in mask construction. A data table follows:

| # of Films | Type of Films | % Transmission |
|---|---|---|
| 0 | None | 100 |
| 4 | 2 Flat/2 Embossed | 60 |
| 3 | 1 Flat/2 Embossed | 62 |
| 1 | 1 Embossed | 82 |
| 2 | 2 Embossed | 72 |
| 1 | 1 Flat | 85 |
| 2 | 2 Flat | 80 |
| 4 | 4 Flat | 60 |

At near infrared frequencies embossed films absorb more light than flat films unless several layers are constructed together. With multiple layers embossed and flat film % Light Transmission is approximately equivalent.

Using a Thwing-Albert Model 323 Digital Opacimeter—with a broad spectrum visible source Bulb DYT: 19V-80 Watt, the following data for % Opacity through various potential designs of film layers useful in mask construction was obtained. A data table follows:

| # of Films | Type of Films | % Opacity |
|---|---|---|
| 3 Nonwovens | 3M Standard Mask | 67.9 |
| 4 | 2 Flat/2 Embossed | 13.3 |
| 3 | 1 Flat/2 Embossed | 12.3 |
| 2 | 2 Embossed | 10.9 |
| 2 | 2 Flat | 4.3 |
| 1 | 1 Embossed | 6.0 |
| 1 | 1 Flat | 2.5 |

Embossed films scatter more visible light than flat films due to the diffraction of the broad light spectrum. For this reason, the preferred embodiment to minimize the opacity of a transparent face mask would consist of flat films separated every other layer by embossed films.

The air porosity for both 3M nonwoven masks and the current application's perforated films are “off scale” for most porosity tests such as Filtrona, Gurley, Sheffield. Porosity using a low head Greiner Porosity Equipment Device which measures in terms of seconds for 100 cc of air flow to pass through the substrate as driven by a water column gave the following results:

| # of Films | Type of Films | Seconds per 100 cc |
|---|---|---|
| 3 Nonwovens | A. 3M Standard Mask | 5.0 |
| 2 | B. 1 Flat/C. 1 Embossed | 4.0 |
| 1 | C. 1 Embossed | 4.0 |
| 1 | B. 1 Flat | 4.0 |

Samples are identified as follows:

### A. 3M Tie Behind Head Surgical Mask;

B. Inside of Mask—(Clear) MicroPerf™ 2 mil PE with 7 (3 mm) vents/inch per & 16 lines/inch or 112 vents/inch 2 or 50% open area; and
C. Outside of Mask—(Embossed) MicroPerf™ 2 mil PE with 7 (3 mm) vents/inch per & 16 lines/inch or 112 vents/inch 2 or 50% open area.

The perforated films, whether used singularly or as double layers have a greater open air porosity than the standard 3M 3 layer nonwoven mask.

An improved transparent face mask is described above. Various details of the invention may be changed without departing from the scope of the invention. Furthermore, the foregoing description of the preferred embodiment of the invention and best mode for practicing the invention are provided for the purpose of illustration only and not for the purpose of limitation, the invention being defined by the claims.

We claim:

1. A mask for being worn over the nose and mouth of a wearer, and including a perimeter frame within which is mounted a panel having sufficient transparency to permit visual observation of the mouth of the wearer, the panel being formed from at least first and second overlying transparent films perforated with openings sized to permit two-way transit of gases but not liquids under atmospheric pressure, at least one of the first and second films including a multitude of projections for contacting the other of the first and second films in a multitude of contact locations for maintaining between the first and second films and the multitude of contact locations a multitude of tortuous flow paths for permitting respiratory gas flow through the panel.

2. A mask according to claim 1, wherein both the first and second films include a multitude of projections, and wherein as least some of the projections on one of the first and second films contact projections on the other of the first and second films.

3. A mask according to claim 1, wherein the projections are formed by embossing.

4. A mask according to claim 1, wherein the openings comprise slits having a length from 1 to 5 mm, the slits being spaced from each other so that the slits define a total open orifice area of approximately 30% to 95.0% of the total surface area of the layer.

5. A mask according to claim 1, wherein the openings comprise holes having a diameter of no more than about 0.127 mm, the holes being spaced from each other so that the holes define a total open orifice area of approximately 30% to 95.0% of the total surface area of the layer.

6. A mask according to claim 1, wherein the first and second films are formed with pleats that are adapted to open into an unpleated configuration during use.

7. A mask according to claim 3, wherein the embossed projections have a height of 0.25 to 1 mm.

8. A mask according to claim 1, wherein one of the films is embossed and one of the films is not embossed.

9. A mask according to claim 1, and including a biocide comprising an organosilane molecules mechanical contact kill mechanism applied to at least one of the films.

10. A mask according to claim 9, wherein the biocide comprises a conventional quaternary ammonium salt (organo) chemically spliced to a silane molecule, resulting in 3-trimethoxysilylpropyldimethyloctadecyl ammonium chloride.

11. A mask for being worn over the nose and mouth of a wearer, and including a perimeter frame within which is mounted a panel having sufficient transparency to permit visual observation of the mouth of the wearer, the panel being formed from first and second overlying transparent, pleated films perforated with elongate slits sized to permit two-way transit of gases but not liquids under atmospheric pressure, the first and second films each including a multitude of projections in a multitude of contact locations for maintaining between the first and second films a multitude of tortuous flow paths for permitting respiratory gas flow through the panel.

12. A mask according to claim 11, wherein the slits have a length of 1 to 5 mm, the slits being spaced from each other so that the slits define a total open orifice area of approximately 30% to 95.0% of the total surface area of the layer.

13. A mask according to claim 11, wherein the embossed projections have a height of 0.25 to 1 mm.

14. A mask according to claim 11, wherein one of the films is embossed and one of the films is not embossed.

15. A mask according to claim 11, and including a biocide comprising an organosilane molecules mechanical contact kill mechanism applied to at least one of the films.

16. A mask according to claim 9, wherein the biocide comprises a conventional quaternary ammonium salt (organo) chemically spliced to a silane molecule, resulting in 3-trimethoxysilylpropyldimethyloctadecyl ammonium chloride.
